(19) United States
(12) Patent Application Publication
(10) Pub. No.: US 2004/0049248 A1
(43) Pub. Date: Mar. 11, 2004

(54) Device for the cleaning wounds by means of laser

(76) Inventor: Karl-Heinz Schonborn, Berlin (DE)

Correspondence Address: CHRISTIE, PARKER & HALE, LLP, 350 WEST COLORADO BOULEVARD, SUITE 500, PASADENA, CA, 91105, US

(21) Appl. No.: 10/415,912

(22) PCT Filed: Nov. 5, 2001
(86) PCT No.: PCT/DE01/04179

(30) Foreign Application Priority Data
Nov. 5, 2000 (DE) 100 55 677.9

(51) Int. Cl. A61N 1/00

(52) U.S. Cl. 607/89

(57) ABSTRACT

The invention relates to a device for the germicidal cleaning of open wounds, characterised by a means for generating at least one pulsed laser beam. It is thus possible to clean wounds efficiently and gently.

### OBJECT OF THE INVENTION

The cleaning of wounds on human or animal accident victims (road traffic, industry and domestic) still represents a major problem.

In the case of open wounds, foreign bodies and dirt frequently enter the open wounds, and can lead to dangerous inflammation. In some cases, foreign bodies are also “tattooed” into the skin surface.

Even after cleaning the wound, there is a risk of germs entering the wound area, and leading to inflammation. The aim must therefore be to remove foreign bodies from the wound, to disinfect the wound and the adjacent skin area, and to maintain this state by means of a suitable dressing.

This preparation for caring for the wound represents the best precondition for promoting rapid healing, without any complications.

Until now, wounds have been cleaned by rinsing and by mechanical cleaning processes, such as wiping or else brushing. These rough methods are complex, cause additional damage to the wounds, and do not provide the necessary freedom from germs in the wound and in the vicinity of the wound.

The fundamental idea of the invention described here is to make use of the particular advantages of laser technology, optical fibers and, in particular, of a fiber-lengthened laser resonator (EP 0 691 043) in order in this case to ensure an advantageous solution to the problem of wound cleaning and disinfection.

Treatment by means of short-pulse laser radiation with a high power density is also a good procedure for reducing the germ load on the tissue, that is to say for achieving a high degree of freedom from germs.

### DESCRIPTION OF THE INVENTION

In order to clean and to reduce the germs in the wound, pulsed laser radiation may also be applied to the adjacent skin area, as well as to the wound surface.

The laser radiation acts specifically on the dirt and foreign bodies, and removes them from the wound by vaporization.

The specific choice of the wavelength and the timing of the pulse profile mean that the wound itself is influenced only to a minor extent. In particular, it is heated only slightly by the apparatus, that is to say only to a maximum of about 50° C., so that a burn injury is not added to an open abrasion or cut wound as a result of thermal effects.

The beam data for the laser radiation is chosen such that the radiation is not absorbed to a major extent in the blood or in the injured tissue, in order to avoid heating it unnecessarily.

It is advantageous to choose laser radiation with short pulses of less than 100 μs, in order that the laser energy vaporizes and removes the dirt particles suddenly by selective absorption, without excessively heating the wound itself. Pulse durations of less than 2.5 μs are, surprisingly, particularly advantageous.

The pulsed laser radiation reduces germs by damaging the cells of the undesirable foreign cells, when it is applied to the tissue. This is an additional desirable effect of the invention.

As an additional measure, the area in which the laser radiation is acting may be supplied with a germ-free rinsing gas, for example with germ-free air or with germ-free inert gas, in order to transport away the decomposition products, and to cool the wound area.

Alternatively, an atomized jet of germ-free rinsing liquid, for example germ-free water or germ-free physiological saline solution, possibly with the addition of antiseptic agents or medicaments that promote healing, may also be used for cooling the wound.

The rinsing agent jet may alternatively be directed synchronized to and aligned with respect to the laser beam onto the wound surface, in order to increase the cleaning effect.

The cooling effect can advantageously be increased by applying rinsing gas or rinsing liquid in an already cooled state, or by adding a cooling agent to the rinsing agent, which produces its cooling effect by vaporization on the wound surface.

A handpiece is preferably used for application, which

1. is supplied with radiation from the laser via an optical fiber or a mirrored hinged arm,

2. collimates the laser radiation, or focuses it onto the target surface, and

3. contains the necessary hose guides and nozzles in order to apply said rinsing and cooling media to the point at which the laser beam is acting.

Preferred Laser Types for Use

A Q-switched Nd:YAG laser, either operated in the pulsed mode or with acousto-optical modulation in the cw mode of the laser, is particularly effective, and at the same time cost-effective. In the lastmentioned case, diode-pumped lasers based on a neodymium-doped laser crystal may also advantageously be used.

As has surprisingly been found, it is particularly advantageous to operate lasers of one of said types, which are operated with the laser line in the range from 1300 to 1550 nm, specifically at the wavelength 1330 nm for Nd:YAG.

The effect can be increased by doubling the frequency, that is to say using a wavelength of 665 nm for an Nd:YAG laser with a fundamental wavelength of 1330 nm.

The selective absorption in dirt particles is in some cases better than 1330 nm, while the absorption in the blood of the open wound is desirably low.

In one particularly advantageous arrangement of the invention, both a portion of the energy at 1330 nm and a portion of the energy at the second harmonic of 665 nm are applied, by means of a suitable configuration of the output mirror, by doubling the frequency of one of said neodymium-doped crystal lasers (preferably Nd:YAG).

The heterogeneous dirt particles absorb predominantly at least at one of the wavelengths, while the absorption in the blood and in the tissue remains moderate at both wavelengths.

These lasers can transmit significant effective energy levels only to a very restricted extent through quartz-glass fibers, so that only direct application or a mirrored hinged arm is feasible.

Fiber-Lengthened Resonator

EP 0 691 043 describes a passively Q-switched laser system whose special feature is an intracavity fiber. This optical fiber is integrated in the laser resonator, thus resulting in a resonator of great length (10-50 m), and a correspondingly long resonator circulation time.

Depending on the configuration, this design results in pulse durations of from 200 ns up to more than 1 μs, which otherwise cannot be achieved either by passively Q-switched lasers or by free running lasers.

In experiments relating to the use of a laser such as this for medical purposes, it has now surprisingly been found that this laser design is particularly advantageous for the indication described here.

If pulse durations of from 200 to 600 ns are used with pulse energies of from 50 to 100 mJ, then this results in sufficiently short and advantageous pulse durations for wound cleaning, while at the same time allowing transmission through quartz-glass fibers.

This leads to an appliance which can be used in a particularly flexible and uncomplicated manner.

With this design, the wavelengths of 1330 and 665 nm result in an advantageous combination, since about 15-30% of the energy is transformed into the second harmonic and is emitted in synchronism with the pulse at the fundamental wavelength.

Beam Data in the Field of Application

In the field of application, that is to say on the surface of the wound or of the injured body surface to be treated, said lasers achieve energy densities of 10 J/mm² or more. Together with the short pulse durations, this results in power densities of 100 kW/mm² or more, preferably of more than 5 MW/mm², in the laser pulse.

In the process, with pulse repetition frequencies of 1 to 100 Hz, the density of the mean power remains moderate, so that the thermal effects are still limited.

The effect on the tissue and the deliberate vaporization of the dirt take place, as a result of the chosen data, primarily by means of photoablative processes or other nonlinear mechanisms that are associated with a high laser power density.

It should, of course, not be forgotten that there is an accompanying positive effect from the biostimulative effect for germ reduction and for promoting healing. This type of influence on metabolism processes and physiological states is normally associated with laser irradiation using a low mean power level in the milliwatt range (low level laser therapy). However, this biostimulative type of effect is not the main aspect of the present invention.

Overall, the apparatus and method described here as the invention can be characterized as “cold” laser wound cleaning and germ reduction.

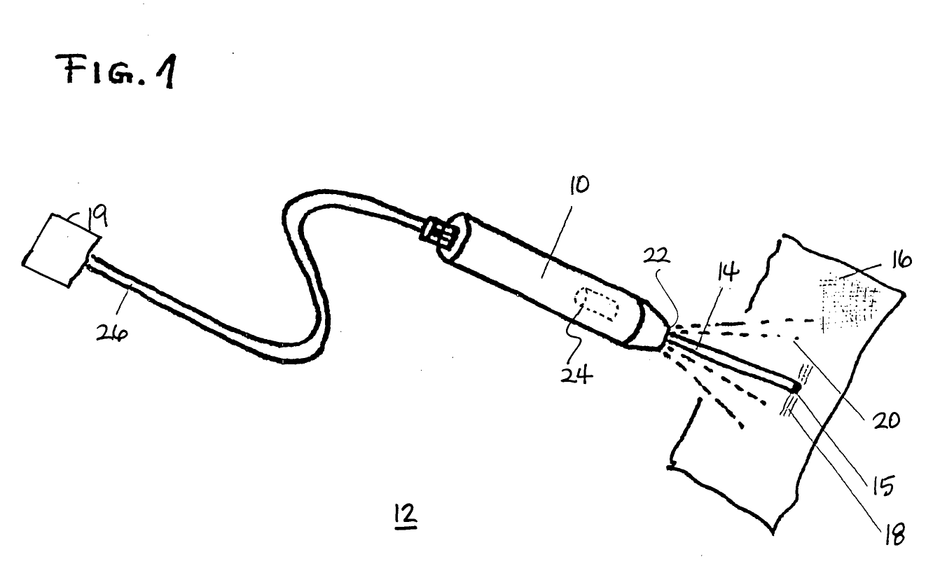

FIG. 1 shows a handpiece for one embodiment of the apparatus according to the invention. In this embodiment, a pulsed laser beam is directed at wounded tissue, with the wound being dirty. The apparatus according to the invention is in this case used to clean the wound. The laser beam is in this case surrounded by jets of a rinsing agent, for example containing a disinfectant agent. Nozzles in this case ensure that it is aligned accurately with the target. Optics and the nozzles for the rinsing agent are arranged in the handpiece itself. The laser light is in this case passed via a cable to the handpiece. Alternatively, the laser can also be produced in the handpiece, provided that a suitable laser is used.

1. An apparatus for cleaning and reducing the germs in open wounds, characterized by a means for producing at least one pulsed laser radiation.

2. The apparatus as claimed in claim 1, characterized in that the means for producing the laser radiation at least partially produces radiation in the wavelength band from 600 to 750 nm.

3. The apparatus as claimed in claim 1 or 2, characterized in that a Q-switched laser is used.

4. The apparatus as claimed in at least one of the preceding claims, characterized in that a solid state laser with frequency doubling is used, preferably an Nd:YAG laser with a fundamental wavelength of 1330 nm, doubled to 665 nm, or some other laser with a neodymium-doped crystal as the laser medium with a fundamental wavelength of 1330±100 nm, doubled to 665±50 nm.

5. The apparatus as claimed in at least one of the preceding claims, characterized in that laser radiation at the fundamental wavelength and at the second harmonic are applied synchronously.

6. The apparatus as claimed in claim 5, characterized in that the energy component of the second harmonic is 15 to 35% of the total energy.

7. The apparatus as claimed in at least one of the preceding claims, characterized in that a Q-switched laser is used with fiber-optic resonator lengthening and with partial or complete frequency doubling with pulse durations in the range from 200 ns to 2 μs, preferably 300 to 600 nm, with a pulse energy of 50 to 100 mJ.

8. The apparatus as claimed in at least one of the preceding claims, characterized in that a flashlamp-pumped laser is used.

9. The apparatus as claimed in at least one of the preceding claims, characterized in that a diode-pumped solid state laser or a diode laser-pumped solid state laser is used.

10. The apparatus as claimed in at least one of the preceding claims, characterized in that the laser radiation is applied by means of a mirrored hinged arm fiber.

11. The apparatus as claimed in at least one of the preceding claims, characterized in that the laser radiation is applied by means of an optical fiber.

12. The apparatus as claimed in at least one of the preceding claims, characterized in that the laser radiation is applied by means of an optical fiber with an optically planar or spherical end surface.

13. The apparatus as claimed in at least one of claims 10 to 12, characterized in that a fiber-optic handpiece, or a handpiece which is fitted to the end of the hinged arm, is used for radiation application.

14. The apparatus as claimed in at least one of the preceding claims, characterized by a means, in particular a handpiece, for supplying a sterile rinsing agent, in particular rinsing gas, under pressure, and nozzles which direct the rinsing gas onto the point at which the laser radiation is acting.

15. The apparatus as claimed in at least one of the preceding claims, characterized in that the means for supplying sterile rinsing agent has a means, in particular nozzles, using which the rinsing agent can be directed onto the point at which the laser beam is acting in the form of a mist, droplets or a jet.

16. The apparatus as claimed in at least one of the preceding claims, characterized in that the rinsing agent or the rinsing gas is supplied in a cooled state.

17. The apparatus as claimed in at least one of the preceding claims, characterized in that a liquid cooling agent, which vaporizes on impact and thus cools the wound, is added to the rinsing agent or to the rinsing gas.

18. The apparatus as claimed in at least one of the preceding claims, characterized in that medicaments or substances which promote healing are added to the rinsing agent or to the rinsing gas and are supplied on impact with the wound surface, where they diffuse in or are reabsorbed in some other way.

19. The apparatus as claimed in at least one of the preceding claims, characterized in that substances which act to reduce germs and reduce the germ load on the wound surface on impact are added to the rinsing agent or to the rinsing gas.
